(19) United States
(12) Patent Application Publication
(10) Pub. No.: US 2024/0371475 A1
(43) Pub. Date: Nov. 7, 2024

(54) ARTIFICIAL INTELLIGENCE-BASED EVALUATION OF DRUG EFFICACY

(71) Applicant: The Regents of the University of California, Oakland, CA (US)

(72) Inventors: Aijun WANG, Sacramento, CA (US); Yongheng Wang, Sacramento, CA (US); Kit LAM, Davis, CA (US); Weidi ZHANG, Sacramento, CA (US)

(73) Assignee: The Regents of the University of California, Oakland, CA (US)

(21) Appl. No.: 18/655,965

(22) Filed: May 6, 2024

Related U.S. Application Data
(60) Provisional application No. 63/500,490, filed on May 5, 2023.

(51) Int. Cl. G16C 20/20; G06V 10/75; G06V 10/82; G06V 20/69

(52) U.S. Cl. CPC G16C 20/20; G06V 10/751; G06V 10/82; G06V 20/698

(57) ABSTRACT

In some aspects, the disclosure is directed to methods and systems for artificial intelligence-based evaluation of pharmaceutical drug efficacy. Disclosed are implementations of deep learning that may be used to analyze histological images, study the differentiation of induced pluripotent stem cells, and perform binary classifications (live or dead, drug treated or untreated) on cancer cells or other target cells. Various type of classifiers or deep learning machines may be used in implementations, along with pre-processing in many implementations to further enhance distinctions between affected and unaffected cells.

### RELATED APPLICATIONS

The present application claims the benefit of and priority to U.S. Provisional Patent Application No. 63/500,490, entitled “Artificial Intelligence-based Evaluation of Drug Efficacy,” filed May 5, 2023, the entirety of which is incorporated by reference herein.

### FIELD OF THE DISCLOSURE

This disclosure generally relates to systems and methods for pharmaceutical analysis. In particular, this disclosure relates to systems and methods for artificial intelligence-based evaluation of pharmaceutical drug efficacy.

### BACKGROUND OF THE DISCLOSURE

As a measure of cytotoxic potency or efficacy of a drug, half-maximal inhibitory concentration (IC50) is the concentration at which a drug exerts half of its maximal inhibitory effect against target cells. Some methods of determining IC50 require applying additional reagents or lysing the cells, and may require significant time and effort. In many methods, cells are destroyed by the reagents, preventing repeated measurements over time.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1A is an illustration of an implementation of a process for determining drug potency or efficacy using MTT;

FIG. 1B is an illustration of an implementation of a process for determining drug potency or efficacy using CCK-8;

FIG. 1C is an illustration of an implementation of a process for determining drug potency or efficacy using ATP;

FIG. 1D is an illustration of an implementation of a process for determining drug potency or efficacy using Hoechst dye;

FIG. 1E is an illustration of an implementation of a convolutional neural network for determining drug potency or efficacy;

FIG. 1F is an illustration of an implementation of a process for determining drug potency or efficacy using a machine learning-based classifier;

FIG. 2A is a block diagram of an implementation of a vision transformer for use in machine learning-based classification and evaluation of drug potency or efficacy;

FIG. 2B is a block diagram of an implementation of an encoding block of a vision transformer for use in machine learning-based classification and evaluation of drug potency or efficacy;

FIG. 3A is an illustration of an example image capture of treated and untreated cells (A) without pre-processing, and (B) with a Gaussian high-pass filter applied, in implementations of machine learning-based classification and evaluation of drug potency or efficacy;

FIG. 3B is an illustration of an example image capture of treated and untreated cells (C) with a Sobel filter, and (D) with an optimized Sobel operator, in implementations of machine learning-based classification and evaluation of drug potency or efficacy;

FIG. 3C is an illustration of graphs of classification accuracies for (E) cephalotaxine, and (f) fasudil, in implementations of machine learning-based classification and evaluation of drug potency or efficacy;

FIG. 3D is an illustration of determining IC50 for various drugs using implementations of machine learning-based classification and evaluation of drug potency or efficacy;

FIG. 3E is a table illustrating differences in IC50s obtained by estimation via Hoechst staining and via implementations of machine learning-based classification and evaluation of drug potency or efficacy;

FIG. 3F is a table illustrating experimental results for various implementations of machine learning-based classification and evaluation of drug potency or efficacy;

FIG. 4 is a block diagram of an implementation of a system for machine learning-based classification and evaluation of drug potency or efficacy;

FIG. 5 is a flow chart of an implementation of a method for machine learning-based classification and evaluation of drug potency or efficacy; and

FIGS. 6A and 6B are block diagrams depicting embodiments of computing devices useful in connection with the methods and systems described herein.

The details of various embodiments of the methods and systems are set forth in the accompanying drawings and the description below.

### DETAILED DESCRIPTION

For purposes of reading the description of the various embodiments below, the following descriptions of the sections of the specification and their respective contents may be helpful:
Section A describes embodiments of systems and methods for artificial intelligence-based evaluation of drug efficacy; and
Section B describes a computing environment which may be useful for practicing embodiments described herein.

### A. Systems and Methods for Artificial Intelligence-Based Evaluation of Drug Efficacy

As a measure of cytotoxic potency or efficacy of a drug, half-maximal inhibitory concentration (IC50) is the concentration at which a drug exerts half of its maximal inhibitory effect against target cells. Some methods of determining IC50 require applying additional reagents or lysing the cells, and may require significant time and effort. In many methods, cells are destroyed by the reagents, preventing repeated measurements over time.

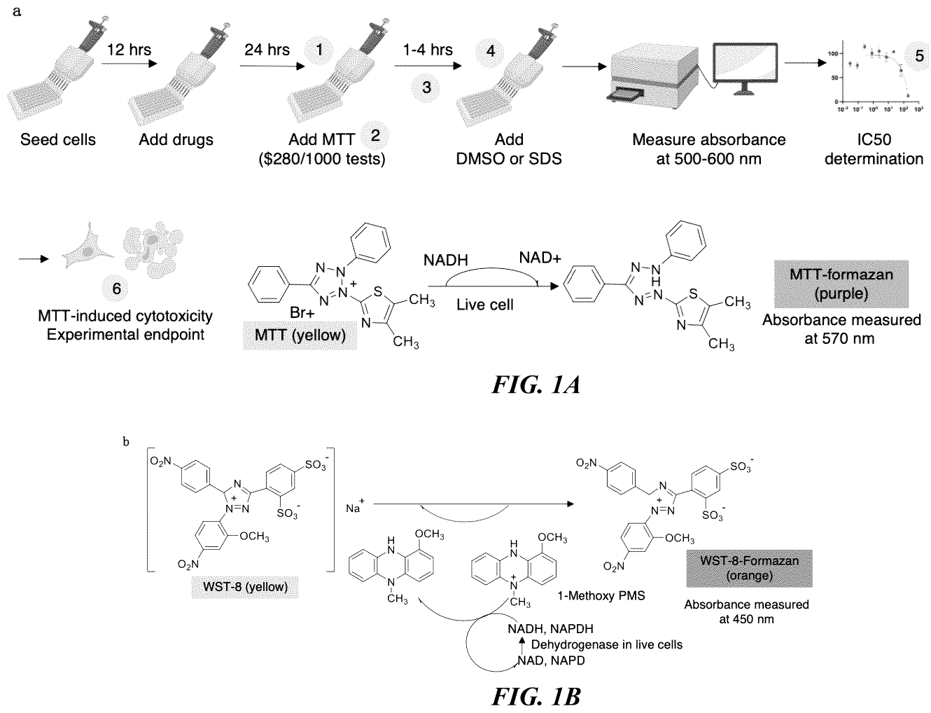

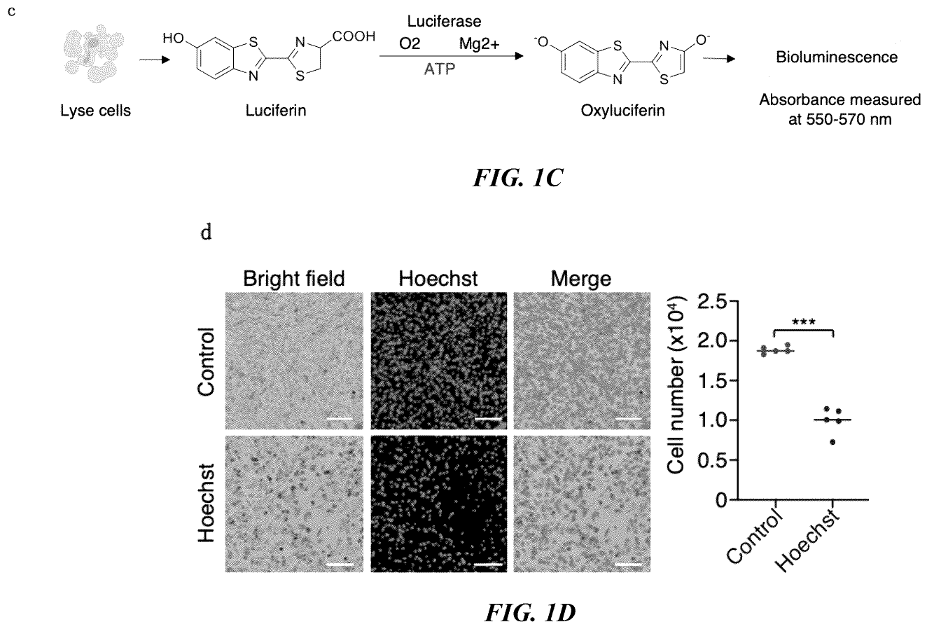

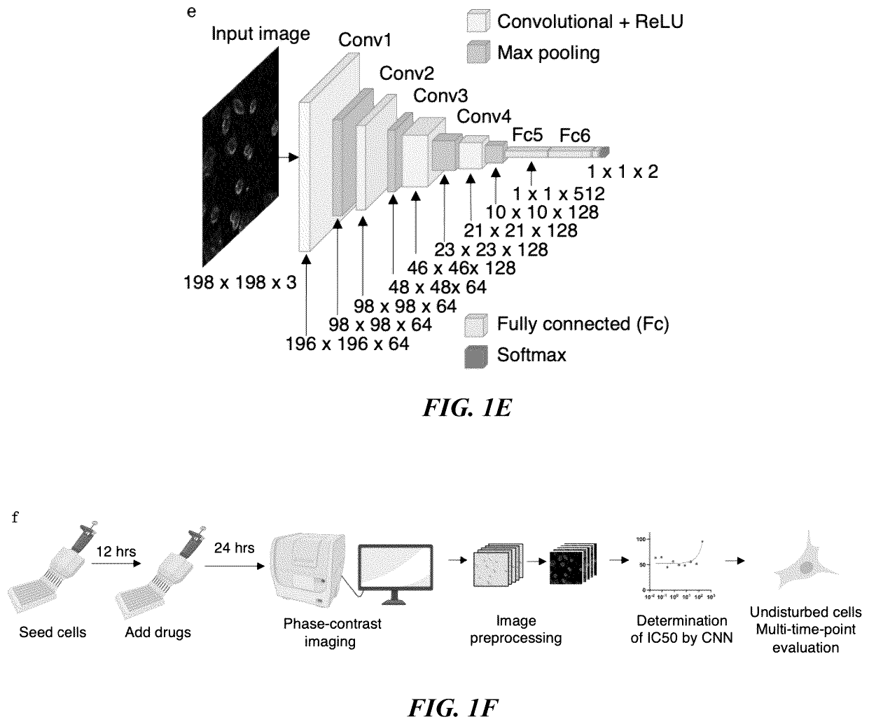

For example, the 3-(4,5-dimethylthiazol-2-yl)-2,5-diphenyl-2H-tetrazolium bromide (MTT) assay has been used to determine the efficacy of drugs. FIG. 1A is an illustration of an implementation of a process for determining drug potency using MTT. Cells are seeded in a multi-well assay plate and grown for a period of time to provide sufficient numbers for measurement, and a drug under examination is applied to the wells in different concentrations and allowed to react with the cells. This may be referred to as initial setup or step 1 in some implementations. At step 2 of the MTT assay, MTT is added to the wells and the positively charged, yellow MTT molecules penetrate viable cells and, over time at step 3, become reduced to purple formazan crystals by mitochondrial dehydrogenase. At step 4, the crystals are then dissolved in dimethyl sulfoxide (DMSO; 100 mL/well in many implementations) or sodium dodecyl sulfate (SDS; 10% w/v in 0.01 M hydrochloric acid, 100 ml/well in many implementations) to become a colored solution with an absorbance maximum near 570 nm, which may be quantified by a spectrophotometer. The absorbance is proportional to the number of live cells, such that the darker the solution, the greater the number of viable cells. Thus, this colorimetric assay measures metabolic activity as an indicator of cell viability at steps 5-6.

Cell counting kit-8 (CCK-8) is another implementation of an assay, and is illustrated in FIG. 1B. It is based on the reduction of water-soluble tetrazolium 8 (WST-8) to 1-methoxy phenazinium methylsulfate (PMS) by nicotinamide adenine dinucleotide (NAD)- or nicotinamide adenine dinucleotide phosphate (NADP)-dependent dihydrogenases in a cell. Compared with MTT, CCK-8 is more sensitive and does not require dissolving solutions.

In an adenosine triphosphate (ATP) assay illustrated in FIG. 1C, cells are lysed to release the ATP, which activates luciferin and yields a luciferyl-adenylate and pyrophosphate. The luciferyl-adenylate reacts with oxygen to generate carbon dioxide and oxyluciferin in an electronically excited state, which releases bioluminescence (550-570 nm) when returned to the ground state. Proportional to the ATP levels and the number of viable cells, the luminescent signal can be quantified to evaluate the inhibitory concentration 50% (IC50) of a cancer drug, the concentration at which half of the cancer cells are killed.

In still another implementation illustrated in FIG. 1D, the IC50 of a drug can also be determined by counting the nuclei stained with Hoechst dye. However, Hoechst staining inhibits the growth of cells.

Instead, the present disclosure is directed to implementations of deep learning that may be used to analyze histological images, study the differentiation of induced pluripotent stem cells, and perform binary classifications (live or dead, drug treated or untreated) on cancer cells or other target cells. Various type of classifiers or deep learning machines may be used in implementations, along with pre-processing in many implementations to further enhance distinctions between affected and unaffected cells.

For example, transformers are a type of self-attention-based deep neural network developed for tasks in the field of natural language processing and employed in computer vision applications thanks to their strong representation capabilities and less need for vision-specific inductive bias. A vision transformer can be built by splitting images into patches, embedding positions, and adding a learnable “classification token.” A transformer encoder typically consists of multiheaded self-attention and multilayer perceptrons (MLPs) containing two layers with Gaussian error linear unit (GELU) non-linearity. Normalization is applied before every block of the encoder to estimate the normalization statistics from the summed inputs to the neurons within a hidden layer.

In a convolutional neural network (CNN), a non-linear activation function is applied to each layer, followed by a max pooling layer to reduce the dimensionality of images and retain the most prominent features. To prevent the neural networks from overfitting, a dropout layer is added between the hidden layer and the output layer. The neuron in fully connected layers applies a linear transformation to the input vector through a weight matrix. A non-linear transformation is then applied to the product through a non-linear activation function, such as a sigmoid function for the binary classification and a softmax function for the multinomial classification.

In various implementations of the systems and methods discussed herein, a vision transformer and CNNs were built to classify preprocessed phase-contrast images and predict the IC50 of drugs. For example, FIG. 1E is an illustration of an implementation of a CNN (Conv2D) that may be utilized in some embodiments of these systems and methods.

In some implementations, a Conv2D neural network is constructed using the TensorFlow framework. The network may include 4 convolutional layers with a rectified linear unit (ReLU). The first two convolutional layers may have 64 kernels, while the third and fourth convolutional layers have 128 kernels in some implementations. At each convolutional layer, the convolution may be performed by sliding the filter over the input data to extract the hidden features from the data. Each convolutional layer may be followed by a 2*2 max pooling layer to reduce the feature dimension by keeping only the most relevant features. The output from the last pooling layer may be flattened and presented to a dropout layer with a rate of 0.5 (or any other suitable value) to avoid overfitting. The last 2 layers in the Conv2D may be fully connected layers with 512 and 2 neurons, respectively, followed by the non-linear activation function to obtain the final classification result. The parameters of the network may be repeatedly trained (e.g. 25, 50, 100, or any other number of epochs) randomly using the training data (e.g. with 90% for training and 10% for testing, or any other such division). The accuracy of the classifier may be then computed by evaluating the fitted model using the test data.

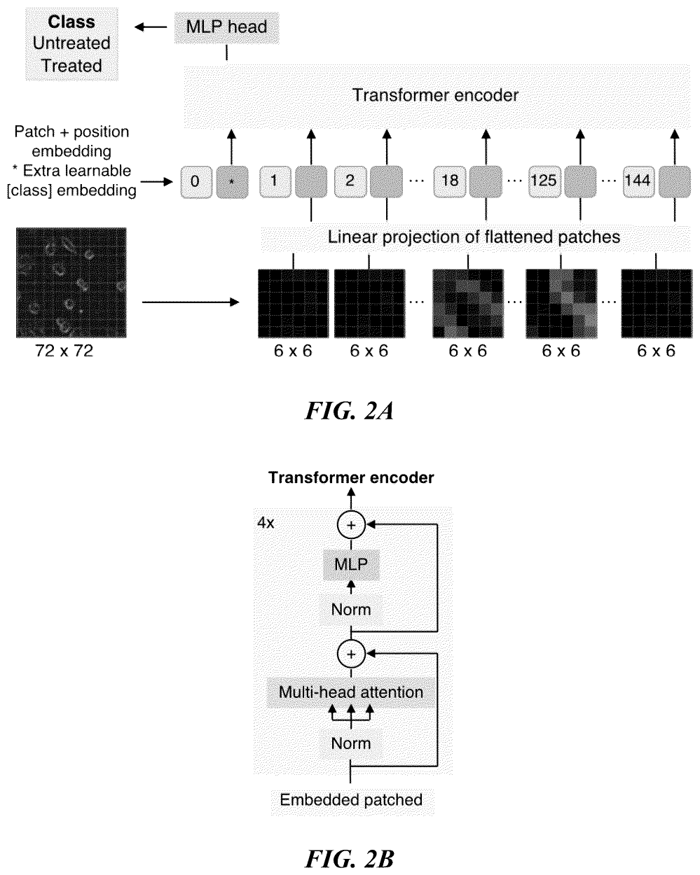

Referring briefly ahead, FIG. 2A is a block diagram of an implementation of a vision transformer for use in machine learning-based classification. Images may be resized to a predetermined size, such as 72×72 pixels, and split into smaller patches (e.g. 6×6 pixels) to build a transformer encoder together with position embeddings and a learnable “classification token,” an implementation of which is shown in the block diagram of FIG. 2B. Layer normalization is implemented before every block of the transformer encoder containing multiheaded self-attention and MLPs. Each MLP may include two layers with GELU non-linearity. In some implementations, hyperparameters may include a learning rate of 0.001 and a weight decay of 0.0001, a batch size of 200, 100 epochs, 4 transformer layers, a dropout rate of 0.1, and MLP head units of [2,048, 1,024], though other values may be used, depending on optimization and desired properties.

Returning to FIG. 1F, illustrated is an implementation of a process for determining drug potency using a machine learning-based classifier.

Compared with the widely used MTT assay discussed above in connection with FIG. 1F (FIG. 1A), implementations of the process of FIG. 1F have one or more of the following advantages: (1) they avoid operational errors and save time associated with adding the reagents; (2) they reduce costs associated with the labeling reagent (i.e., MTT), balancing buffer, filters, and dissolving solutions; (3) they do not require incubation time; (4) they can be used to screen a broader range of chemicals because compounds with absorbance from 450 to 600 nm or with antioxidant properties will interfere with the MTT or CCK-8 absorbance measurement; (5) the cells do not need to be in the log phase, unlike MTT assays which may require cells to be in the log phase to ensure the linearity between absorbances and cell numbers; and (6) the process is not cytotoxic, permitting multi-time point measurements.

To verify operation of implementations of the systems and methods discussed herein, melanoma cells were treated with different drugs at various concentrations. Data was then collected using a high-throughput automated imaging system (e.g., Pico manufactured by ImageXpress, Cytation 5 manufactured by BioTek, or any other cell imaging system). Multiple images were captured for each drug and separated into training (e.g. 90%) and testing (e.g. 10%) pools (e.g. more than 2000 images for training purposes and 200 images for testing, in some implementations), as shown in the table below:
Drug: Training images/Test images
Paclitaxel: 4,320/480
Cephalotaxine: 5,040/560
Fasudil: 5,600/600
Irinotecan: 4,680/520

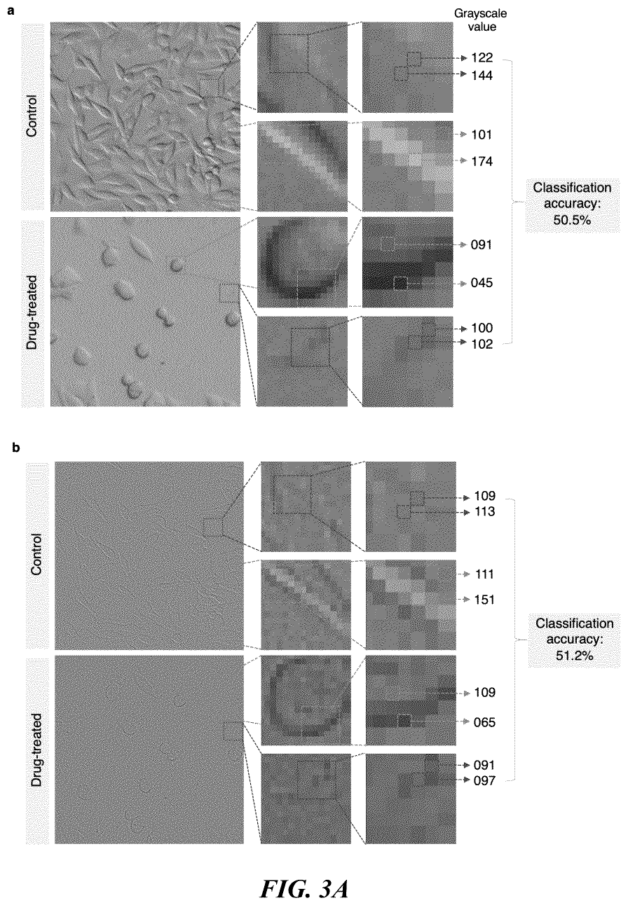

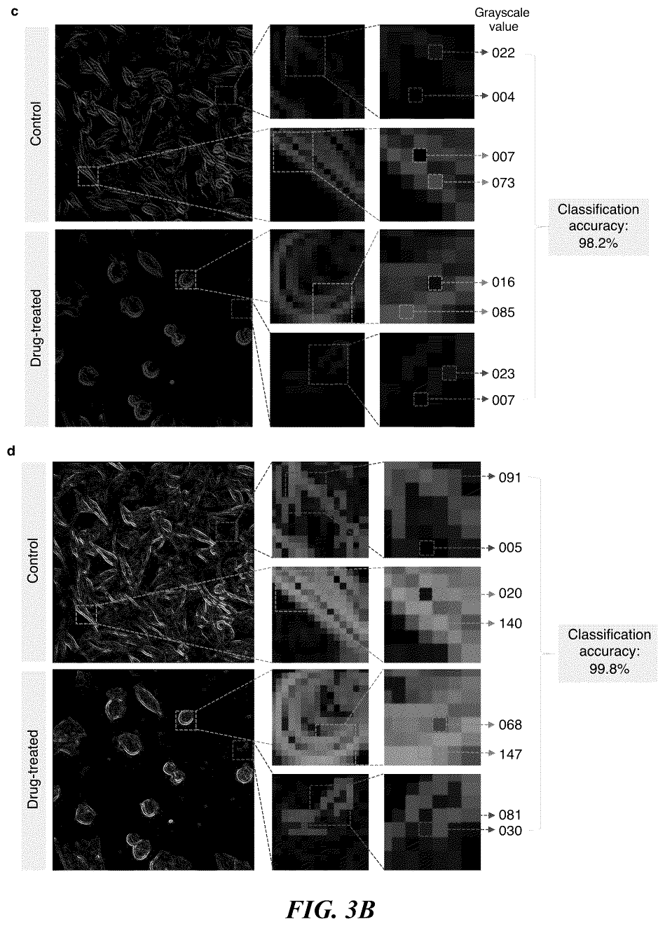

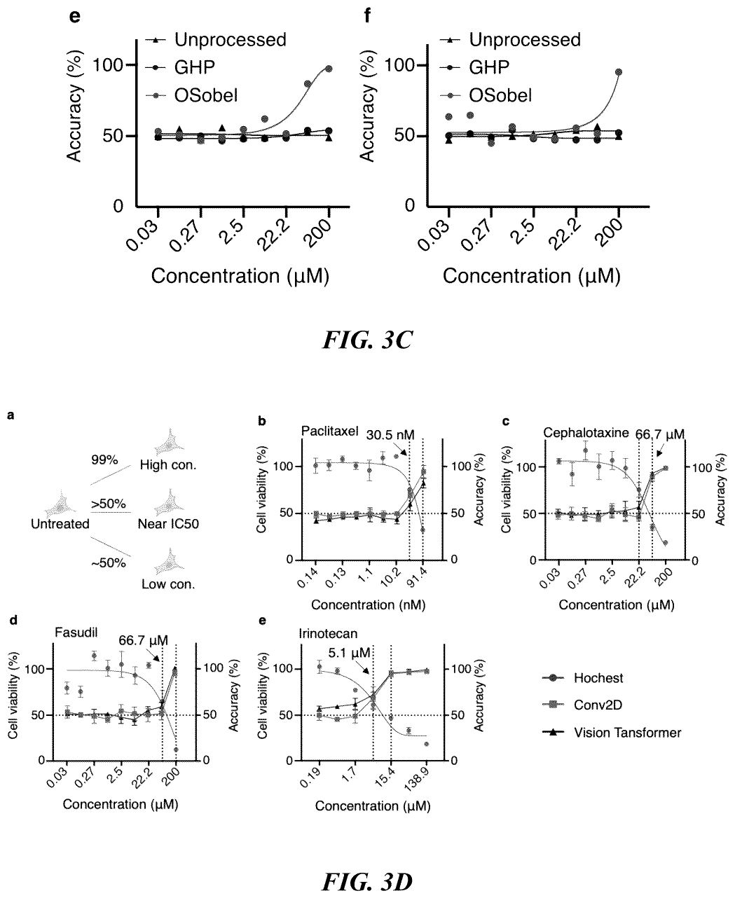

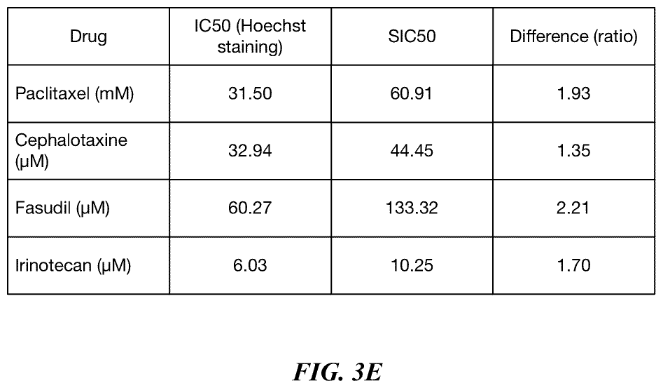

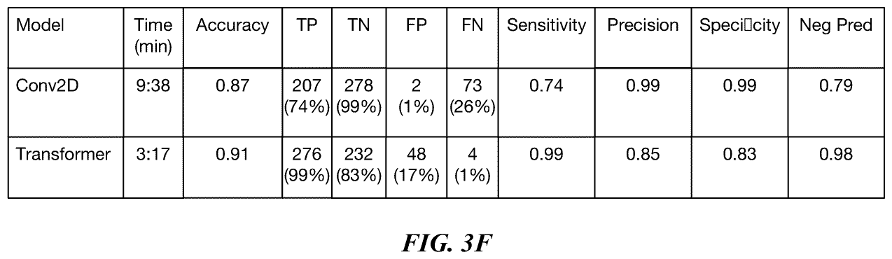

For example, the training folder for paclitaxel contains 4,320 images, which are evenly divided between the treated and untreated groups. The testing folder has 480 images (half from the treated group and half from the untreated group), accounting for 10% of the total paclitaxel dataset. Each image contains various numbers of cells with different densities and morphological features. For example, in the image capture A of FIG. 3A, B16-F10 cells were treated with 200 mM paclitaxel for 24 hours. Scale bars are 15 μm. The raw image is a 2007×2007 16-TIFF file and was converted to an 8-bit PNG and split into 100 patches. The images were augmented by a 40 degree rotation and a horizontal flip to provide additional versions. The pixel values (0-255) of the images were divided by 255 to rescale the data. After data preprocessing, binary classification was conducted using untreated cells as the control.

In image A of FIG. 3A, images are not pre-processed, and classification accuracies are low. CNNs cannot accurately classify untreated and treated cells before preprocessing images (accuracy=50.5%). The RGB values of the background are large and relatively close to that of cells. Image B of FIG. 3A shows an implementation that includes pre-processing the images with a Gaussian high-pass (GHP) filter. However, as with the implementation of image A, accuracy is not significantly improved (accuracy=51.2%). The difference between the background and the signal is small.

Conversely, FIG. 3B is an illustration of an example image capture of treated and untreated cells (C) with a Sobel filter, and (D) with an optimized Sobel operator, in implementations of machine learning-based classification. In image C of FIG. 3B, a Sobel filter is applied to the images prior to classification, reducing the grayscale values in the background, increasing the signal-to-noise ratio, and improving prediction accuracy to 98.2%. Similarly, in image D of FIG. 3B, an optimized Sobel operator (OSobel) is used to pre-process images, resulting in a classification accuracy of 99.8%. Although primarily discussed in connection with Sobel filters, any other processing algorithm that can increase the signal to noise ratio of the images may be utilized (e.g. edge detection algorithms including Canny edge detection or differential edge detection, Laplacian filters, etc.).

FIG. 3C is an illustration of graphs of classification accuracies for (E) cephalotaxine, and (F) fasudil, in implementations of machine learning-based classification. As shown, and similar to the results illustrated in FIGS. 3A and 3B, pre-processing the images with the optimized Sobel operator results in significant increases in classification accuracy at higher drug concentrations. As shown in FIG. 3C, regardless of the preprocessing methods, the accuracies are equally poor at low concentrations for treated cells. However, when the cells are treated with drugs (e.g., cephalotazine and fasudil) at high concentrations, the classification accuracies using images preprocessed with an OSobel algorithm or other processor are higher than that of Gaussian high-pass and unprocessed images.

The accuracy of the binary classification can be used to predict the IC50 of a drug. To show efficacy of the systems and methods discussed herein, the cell viability and IC50 of drugs were first determined using Hoechst staining and are represented as blue dots in graphs B-E of FIG. 3D. For example, cells were incubated with culture media containing 1 mg/ml Hoechst 33342 (Thermo Fisher Scientific, cat. no. H3570), for 10 min. The IC50 was calculated after counting the number of nuclei with CellProfiler and curve fitting with GraphPad Prism 9. The IC50 approximately equals the x axis value of the intersecting point of a blue curve in images B-E of FIG. 3D and the horizontal dashed line crossing the y axis at 50%.

Binary classifications were performed using untreated cells and the cells treated with drugs at various concentrations as shown in image A of FIG. 3D. The classification accuracies at different concentrations are labeled as red squares in each of graphs B-E. As shown, the accuracies of binary classification are close to 50% when the cells are treated with drugs at low concentrations, suggesting that low concentrations of the drugs do not result in detectable changes in cell density or cellular morphology. When drawing lines connecting every pair of adjacent red squares, the line with the highest slope contains values close to the IC50s of drugs (graphs B-E of FIG. 3D, and labeled with vertical dotted lines through each adjacent measurement point). Although the average value of these two adjacent concentrations determined via the systems and methods discussed herein can be different from the IC50s estimated using Hoechst staining by 2-fold, mapping the IC50 from a large range of concentrations (0.03-200 mM, 6,666-fold) to a 2-fold variation can be helpful and sufficient for many purposes. For example, FIG. 3E is a table illustrating differences in IC50s obtained by estimation via Hoechst staining and via implementations of machine learning-based classification (referred to as “SIC50”). In some implementations, 1.5- or 1.25-fold dilutions could be adopted to further improve the prediction accuracy of SIC50. The vision transformer may also perform better when pretrained at a large scale and transferred to tasks with fewer images.

Accordingly, in many implementations, the system may determine a plurality of classification accuracies at different concentrations of a candidate drug and determine a point or region (e.g. between two neighboring concentration amounts) of greatest increase in accuracy. This may be determined in any suitable way, such as by fitting a function or curve to the determined classification accuracies, calculating a derivative of the function or curve, and determining a highest value of the derivative. In another implementation, this may be done by calculating a slope between each adjacent or neighboring pair of concentration amounts. For example, given test concentrations of 0.1 μM, 0.2 μM, 0.3 μM, 0.4 μM, etc., and corresponding classification accuracies of 50%, 52%, 55%, 70%, etc., the system may calculate a slope between the determined adjacent pairs of accuracies as y2−y1/x2−x1 (e.g. 2%/0.1 μM from 0.1 μM to 0.2 M; 3%/0.1 μM from 0.2 μM to 0.3 μM; 15%/0.1 μM from 0.3 μM to 0.4 μM; etc. (in many implementations, units may be disregarded or left out)). This may be faster in many implementations than fitting a polynomial function, though it may be less accurate than a derivative-based determination, depending on spacing of test concentrations. In another implementation, a non-linear regression curve may be utilized and a Hill equation applied, such as Y=Min+(Max−Min)/(1+(X/IC50)\hat{ }H) with Max and Min being the maximum and minimum values of optical density at a given wavelength (e.g. optical density at 450 nm or OD_{450 nm}), and H being a Hill coefficient. In other implementations, other methods of finding the greatest accuracy change may be utilized. Although the example above uses equally separated concentration values, in many implementations, concentration ranges may be non-linear (for example, as shown in the examples of FIG. 3C).

To compare the performance of the deep-learning models, two experiments were conducted. First, a small number of images (n=100) were used to train both models. In the results shown in the table FIG. 3F, implementations using a vision transformer are faster and more accurate than those using Conv2D (“TP” is true positives; “FP” is false positives; “FN” is false negatives; “FP” is false positives”, and “Neg pred” is negative predictive). In a further experiment, the accuracy of Conv2D was improved to be comparable to the accuracy of the vision transformer when a slightly larger dataset (n=250) was used for training. The transformer implementations were still significantly faster, which may be desirable in many instances.

The SIC50 models were tested using drugs with different mechanisms of action. For example, paclitaxel stabilizes microtubules, increases microtubule polymerization, decreases microtubule depolymerization, prevents mitosis, and blocks cell-cycle progression. Cephalotaxin inhibits the growth of cancer cells by activating the mitochondrial apoptosis pathway. Fasudil is a calcium channel blocker and inhibits the Rho-kinase signaling pathway. Irinotecan is a prodrug of 7-ethyl-10-hydroxycamptothecin (SN-38), which forms complexes with topoisomerase 1B and DNA and causes DNA misalignment and cell death. Accordingly, implementations of the methods and systems discussed herein can be used for screening different categories of drugs, and the example drugs identified herein are not to be considered exhaustive or to exclude other drugs.

Implementations of the systems and methods discussed herein will empower drug discovery and research in pharmacology by facilitating the high-throughput screening of chemical libraries using 1,536-well (or larger) plates and imaging platforms such as the Cytation 5, helping evaluate the potency of other small-molecule drugs, small interfering RNA (siRNA), and microRNA and assessing the cytotoxicity of delivery vehicles for drugs and genes such as lipid nanoparticles, polymers, and adeno-associated viruses. In addition, implementations of these methods and systems can be modified to facilitate biomedical research related to changes in cellular morphology, e.g., cancer cell metastasis, stem cell differentiation, neural plasticity, and so on.

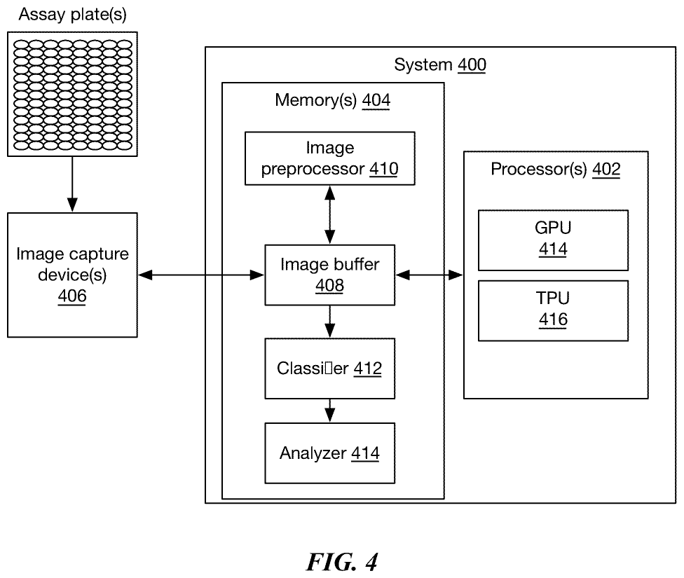

FIG. 4 is a block diagram of an implementation of a system 400 for machine learning-based classification and evaluation of drug potency or efficacy. System 400, which may comprise any type or form of computing device or combination or collection of computing devices, may comprise one or more processors 402, including central processing units and co-processors such as graphics processing units (GPU) 414 and/or tensor processing units (TPU) 416; and one or more memory devices 404, such as flash memory, hard drives, network attached storage, external storage devices, etc. System 400 may comprise or communicate with an image capture device 406, which may capture images of treated and untreated cells in wells of an assay plate. In some implementations, system 400 may comprise physical computing devices, virtual computing devices, or a combination of physical and virtual computing devices (e.g. a server cloud of virtual computing device executed by one or more physical computing devices). Accordingly, though illustrated as a single device, in many implementations, system 400 may comprise a plurality of devices.

In some implementations, memory 404 may comprise an image buffer 408, which may comprise storage for holding and processing captured images. Memory 404 may comprise an image preprocessor 410, which may execute an edge detection algorithm, Sobel operator, optimized Sobel operator, or any other such processing algorithms. In some implementations, preprocessor 410 may be used to augment captured images, e.g. via scaling, translation, and/or rotation, to build up a larger data set for training and/or testing purposes. Memory 404 may comprise a classifier 412. Memory 404 may also comprise an analyzer 414 for identifying an IC50 based on accuracy of the classifier at various concentrations of a drug under test.

Classifier 412 may comprise a convolutional neural network, vision transformer, or any other suitable type of classifier as discussed above. Classifier 412 may be embodied in hardware, software, or a combination of hardware and software. For example, classifier 412 may be executed by a tensor processing unit or similar co-processor executing a machine-learning model for classifying processed images as treated or untreated. As discussed above, classifier 412 may be trained via a supervised learning process on a subset of data comprising treated and untreated cells at different known concentrations.

Analyzer 414 may comprise an application, service, server, daemon, routine, or other executable logic for determining an IC50. In some implementations, analyzer 414 may determine a classification accuracy or confidence level of a classification by classifier 412. In some implementations, classifier 412 may provide an indication of confidence or accuracy of a classification, while in other implementations, analyzer 414 may determine a confidence or accuracy based on a comparison of classifications of images of treated and untreated cells to known truth values and/or manually determined classifications of the corresponding images.

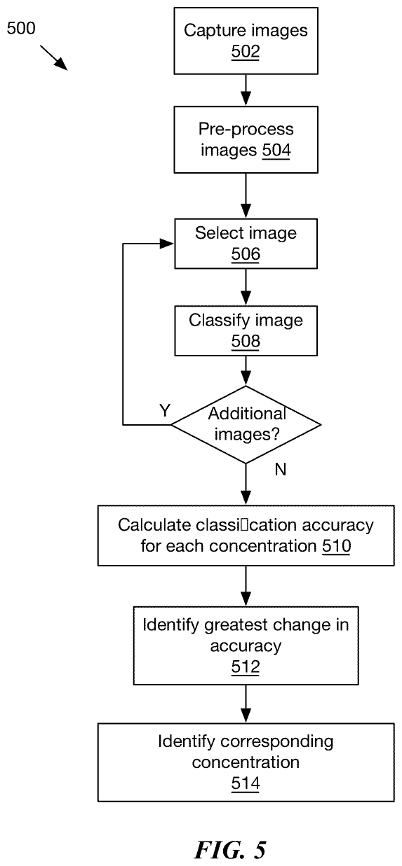

FIG. 5 is a flow chart of an implementation of a method for machine learning-based classification and evaluation of drug potency or efficacy. At step 502, data may be gathered from cultured cells at different concentrations of a drug, e.g. via images captured from an assay plate after a period of incubation, such as bright field imaging. The data may comprise a first plurality of images of untreated cells and a second plurality of images of cells treated with a candidate drug, the second plurality of images comprising subsets of one or more images, each subset corresponding to a different concentration of the candidate drug. The subsets may be referred to variously as concentration subsets, concentration buckets, image buckets, image groups, or by similar terms. Each subset may be considered to be adjacent to one or two other subsets having a next-higher or next-lower concentration (i.e. with the lowest and highest subsets adjacent to only one other subset). For example, given subsets corresponding to concentrations of 10 ppm, 50 ppm, 100 ppm, and 200 ppm, the 50 ppm-associated subset may be considered to be adjacent to or neighboring the 10 ppm-associated subset and 100 ppm-associated subset. Similarly, the 100 ppm-associated subset may be considered to be adjacent to or neighboring the 50 ppm-associated subset and the 200 ppm-associated subset. In some implementations, adjacent subsets may be identified as a positive adjacent subset (i.e. a subset associated with a next-highest concentration) or a negative adjacent subset (i.e. a subset associated with a next-lower concentration).

In some implementations, cell viability may be determined via manual count (e.g. CellProfiler, as discussed above) to provide a baseline for determining accuracy of the classifier. Each image may be associated with a concentration and a measured viability. In some implementations, the measured viability may not be a count, but rather an identifier of viable or non-viable (or that the drug was effective or non-effective, or that cellular activity was inhibited or not-inhibited), and thus may be referred to as a determined viability, a determined inhibitory function, or any other similar term.

At step 504 in some implementations, the data may be pre-processed. Pre-processing the data may include dividing the input data into training data and test data (e.g. 90% training data and 10% test data or any other such ratio). In some implementations, pre-processing the data may also comprise augmenting the data (e.g. creating copies of the input images with one or more manipulations, such as one or more of scaling, normalization, rotation, resolution reduction, flipping, random cropping, etc.—such copies may be associated with the measured viability of the original version). In some implementations, brightness, contrast, saturation, and/or hue may be adjusted for each image to predetermined ranges. In some implementations, pre-processing the data may comprise applying a filter, such as a Sobel filter or other edge detection algorithm. In other implementations, this may be performed at step 508.

Images may be selected at step 506 and classified at step 508, with the process repeated for each additional image. In some implementations, step 508 may be performed in parallel. For example, the data may be divided amongst one or more processors, appliances, servers, or other devices for analysis and processing in some implementations, allowing for scalability. Classification may comprise processing each image via a supervised learning algorithm, such as a neural network or other classifier discussed above, with the pre-processed and/or filtered images as input and the measured or determined viability as output (e.g. treated or untreated, viable or non-viable, etc.). Regression learning may be used to adjust weights or other hyperparameters to improve accuracy of the classification.

Once trained, the model may be applied to the pre-processed test data at steps 506-508. The classification accuracy of the model at each concentration may be determined (e.g. as total correct classifications divided by the total number of classifications, or any similar metric) at step 510. In some implementations, if classification accuracy is too low at every concentration (e.g. below a threshold), additional training may be performed.

At step 512, the system may determine a greatest change or increase in classification accuracy between neighboring or adjacent concentration values. This may be done via fitting a regression curve and applying a Hill equation, determining a derivative of a form fitting curve, calculating a slope between neighboring discrete points, or any other method. The greatest or maximum increase in accuracy may be identified as corresponding to the IC50 at step 514. In some implementations, the change or increase may be determined relative to a positive adjacent subset (i.e. a next-highest concentration-associated subset), a negative adjacent subset (i.e. a next-lowest concentration-associated subset), or both a positive and a negative adjacent subset.

Accordingly, the systems and methods discussed herein provide implementations of deep learning that may be used to analyze histological images, study the differentiation of induced pluripotent stem cells, and perform binary classifications (live or dead, drug treated or untreated) on cancer cells or other target cells. Various type of classifiers or deep learning machines may be used in implementations, along with pre-processing in many implementations to further enhance distinctions between affected and unaffected cells.

In a first aspect, the present disclosure is directed to a method for determining drug inhibitory concentrations. The method includes receiving, by one or more computing devices, a plurality of images of cells treated with a candidate drug, the plurality of images comprising subsets of one or more images, each subset corresponding to a different concentration of the candidate drug. The method also includes classifying, by the one or more computing devices, each image of the plurality of images as treated or untreated. The method also includes calculating, by the one or more computing devices, a classification accuracy for each subset of the plurality of images. The method also includes determining, by the one or more computing devices, a concentration of the candidate drug corresponding to a subset of the plurality of images having a greatest change in classification accuracy relative to a subset corresponding to a next-highest or next-lowest concentration. The method also includes identifying, by the one or more computing devices, the determined concentration as corresponding to an inhibitory concentration.

In some implementations, the method includes receiving, by the one or more computing devices, a second plurality of images of untreated cells. In a further implementation, the method includes dividing the plurality of images of treated cells and the second plurality of images of untreated cells into a first set of training data and a second set of test data.

In some implementations, the method includes augmenting the plurality of images of cells treated with the candidate drug by creating additional images via one or more image manipulations of the received images. In some implementations, the method includes filtering, by the one or more computing devices, each image of the plurality of images to identify edges within each image. In a further implementation, filtering each image of the plurality of images includes applying a Sobel filter to each image.

In some implementations, the method includes classifying each image of the plurality of images as treated or untreated by providing each image to one or more vision transformers executed by the one or more computing devices.

In some implementations, the method includes calculating the classification accuracy for each concentration of the candidate drug by comparing the classification of each image to a predetermined treatment classification for the image.

In some implementations, the method includes determining the concentration of the candidate drug corresponding to the subset of the plurality of images having the greatest change in classification accuracy relative to a subset corresponding to a next-highest or next-lowest concentration by determining a Hill curve corresponding to the calculated classification accuracies; and identifying a concentration of the candidate drug corresponding to a portion of the Hill curve having a greatest slope.

In some implementations, the method includes determining the concentration of the candidate drug corresponding to the subset of the second plurality of images having the greatest change in classification accuracy relative to a subset corresponding to a next-highest or next-lowest concentration by determining a slope between each pair of adjacent concentrations and corresponding classification accuracies and identifying a concentration of the candidate drug corresponding to a maximum determined slope.

In another aspect, the present disclosure is directed to a system for determining drug inhibitory concentrations. The system includes one or more computing devices comprising one or more processors configured to receive a plurality of images of cells treated with a candidate drug, the plurality of images comprising subsets of one or more images, each subset corresponding to a different concentration of the candidate drug. The one or more processors are also configured to classify each image of the plurality of images as treated or untreated. The one or more processors are also configured to calculate a classification accuracy for each subset of the plurality of images. The one or more processors are also configured to determine a concentration of the candidate drug corresponding to a subset of the plurality of images having a greatest change in classification accuracy relative to a subset corresponding to a next-highest or next-lowest concentration. The one or more processors are also configured to identify the determined concentration as corresponding to an inhibitory concentration.

In some implementations, the one or more processors are further configured to receive a second plurality of images of untreated cells. In a further implementation, the one or more processors are further configured to divide the plurality of images of treated cells and the second plurality of images of untreated cells into a first set of training data and a second set of test data.

In some implementations, the one or more processors are further configured to augment the plurality of images of cells treated with the candidate drug by creating additional images via one or more image manipulations of the received images. In some implementations, the one or more processors are further configured to filter each image of the plurality of images to identify edges within each image. In a further implementation, filtering each image of the plurality of images further comprises applying a Sobel filter to each image.

In some implementations, the one or more processors are further configured to apply one or more vision transformers to each image. In some implementations, the one or more processors are further configured to compare the classification of each image to a predetermined treatment classification for the image.

In some implementations, the one or more processors are further configured to determine a Hill curve corresponding to the calculated classification accuracies; and identify a concentration of the candidate drug corresponding to a portion of the Hill curve having a greatest slope.

In some implementations, the one or more processors are further configured to determine a slope between each pair of adjacent concentrations and corresponding classification accuracies and identify a concentration of the candidate drug corresponding to a maximum determined slope.

### B. Computing Environment

Having discussed specific embodiments of the present solution, it may be helpful to describe aspects of the operating environment as well as associated system components (e.g., hardware elements) in connection with the methods and systems described herein.

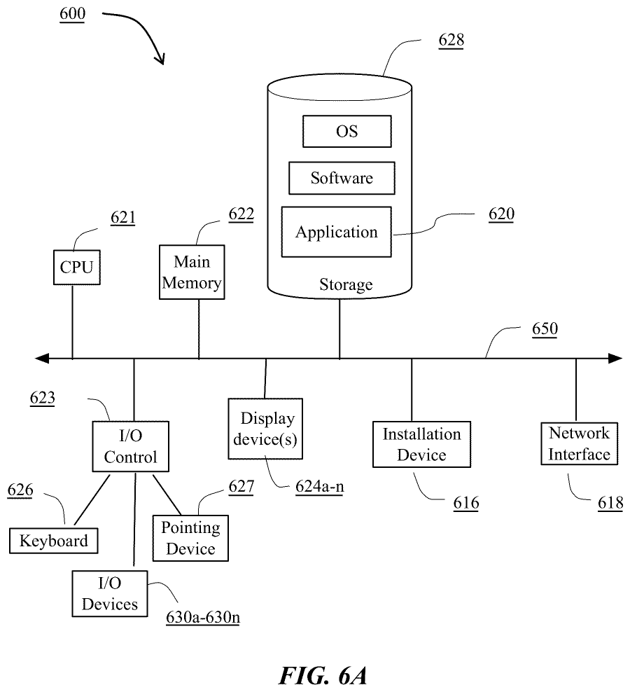

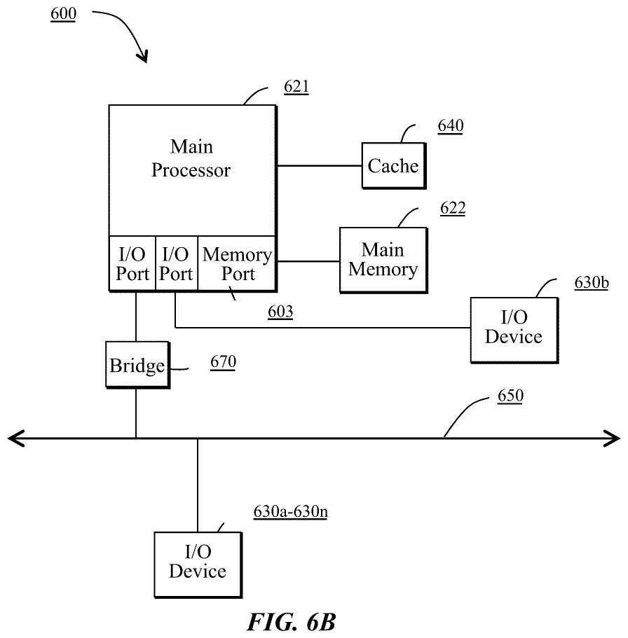

The systems discussed herein may be deployed as and/or executed on any type and form of computing device, such as a computer, network device or appliance capable of communicating on any type and form of network and performing the operations described herein. FIGS. 6A and 6B depict block diagrams of a computing device 600 useful for practicing an embodiment of the wireless communication devices 602 or the access point 606. As shown in FIGS. 6A and 6B, each computing device 600 includes a central processing unit 621, and a main memory unit 622. As shown in FIG. 6A, a computing device 600 may include a storage device 628, an installation device 616, a network interface 618, an I/O controller 623, display devices 624a-624n, a keyboard 626 and a pointing device 627, such as a mouse. The storage device 628 may include, without limitation, an operating system and/or software. As shown in FIG. 6B, each computing device 600 may also include additional optional elements, such as a memory port 603, a bridge 670, one or more input/output devices 630a-630n (generally referred to using reference numeral 630), and a cache memory 640 in communication with the central processing unit 621.

The central processing unit 621 is any logic circuitry that responds to and processes instructions fetched from the main memory unit 622. In many embodiments, the central processing unit 621 is provided by a microprocessor unit, such as: those manufactured by Intel Corporation of Mountain View, California; those manufactured by International Business Machines of White Plains, New York; or those manufactured by Advanced Micro Devices of Sunnyvale, California. The computing device 600 may be based on any of these processors, or any other processor capable of operating as described herein.

Main memory unit 622 may be one or more memory chips capable of storing data and allowing any storage location to be directly accessed by the microprocessor 621, such as any type or variant of Static random access memory (SRAM), Dynamic random access memory (DRAM), Ferroelectric RAM (FRAM), NAND Flash, NOR Flash and Solid State Drives (SSD). The main memory 622 may be based on any of the above described memory chips, or any other available memory chips capable of operating as described herein. In the embodiment shown in FIG. 6A, the processor 621 communicates with main memory 622 via a system bus 650 (described in more detail below). FIG. 6B depicts an embodiment of a computing device 600 in which the processor communicates directly with main memory 622 via a memory port 603. For example, in FIG. 6B the main memory 622 may be DRDRAM.

FIG. 6B depicts an embodiment in which the main processor 621 communicates directly with cache memory 640 via a secondary bus, sometimes referred to as a backside bus. In other embodiments, the main processor 621 communicates with cache memory 640 using the system bus 650. Cache memory 640 typically has a faster response time than main memory 622 and is provided by, for example, SRAM, BSRAM, or EDRAM. In the embodiment shown in FIG. 6B, the processor 621 communicates with various I/O devices 630 via a local system bus 650. Various buses may be used to connect the central processing unit 621 to any of the I/O devices 630, for example, a VESA VL bus, an ISA bus, an EISA bus, a MicroChannel Architecture (MCA) bus, a PCI bus, a PCI-X bus, a PCI-Express bus, or a NuBus. For embodiments in which the I/O device is a video display 624, the processor 621 may use an Advanced Graphics Port (AGP) to communicate with the display 624. FIG. 6B depicts an embodiment of a computer 600 in which the main processor 621 may communicate directly with I/O device 630b, for example via HYPERTRANSPORT, RAPIDIO, or INFINIBAND communications technology. FIG. 6B also depicts an embodiment in which local busses and direct communication are mixed: the processor 621 communicates with I/O device 630a using a local interconnect bus while communicating with I/O device 630b directly.

A wide variety of I/O devices 630a-630n may be present in the computing device 600. Input devices include keyboards, mice, trackpads, trackballs, microphones, dials, touch pads, touch screen, and drawing tablets. Output devices include video displays, speakers, inkjet printers, laser printers, projectors and dye-sublimation printers. The I/O devices may be controlled by an I/O controller 623 as shown in FIG. 6A. The I/O controller may control one or more I/O devices such as a keyboard 626 and a pointing device 627, e.g., a mouse or optical pen. Furthermore, an I/O device may also provide storage and/or an installation medium 616 for the computing device 600. In still other embodiments, the computing device 600 may provide USB connections (not shown) to receive handheld USB storage devices such as the USB Flash Drive line of devices manufactured by Twintech Industry, Inc. of Los Alamitos, California.

Referring again to FIG. 6A, the computing device 600 may support any suitable installation device 616, such as a disk drive, a CD-ROM drive, a CD-R/RW drive, a DVD-ROM drive, a flash memory drive, tape drives of various formats, USB device, hard-drive, a network interface, or any other device suitable for installing software and programs. The computing device 600 may further include a storage device, such as one or more hard disk drives or redundant arrays of independent disks, for storing an operating system and other related software, and for storing application software programs such as any program or software 620 for implementing (e.g., configured and/or designed for) the systems and methods described herein. Optionally, any of the installation devices 616 could also be used as the storage device. Additionally, the operating system and the software can be run from a bootable medium.

Furthermore, the computing device 600 may include a network interface 618 to interface to the network 604 through a variety of connections including, but not limited to, standard telephone lines, LAN or WAN links (e.g., 802.11, T1, T3, 56 kb, X.25, SNA, DECNET), broadband connections (e.g., ISDN, Frame Relay, ATM, Gigabit Ethernet, Ethernet-over-SONET), wireless connections, or some combination of any or all of the above. Connections can be established using a variety of communication protocols (e.g., TCP/IP, IPX, SPX, NetBIOS, Ethernet, ARCNET, SONET, SDH, Fiber Distributed Data Interface (FDDI), RS232, IEEE 802.11, IEEE 802.11a, IEEE 802.11b, IEEE 802.11g, IEEE 802.11n, IEEE 802.11ac, IEEE 802.11ad, CDMA, GSM, WiMax and direct asynchronous connections). In one embodiment, the computing device 600 communicates with other computing devices 600′ via any type and/or form of gateway or tunneling protocol such as Secure Socket Layer (SSL) or Transport Layer Security (TLS). The network interface 618 may include a built-in network adapter, network interface card, PCMCIA network card, card bus network adapter, wireless network adapter, USB network adapter, modem or any other device suitable for interfacing the computing device 600 to any type of network capable of communication and performing the operations described herein.

In some embodiments, the computing device 600 may include or be connected to one or more display devices 624a-624n. As such, any of the I/O devices 630a-630n and/or the I/O controller 623 may include any type and/or form of suitable hardware, software, or combination of hardware and software to support, enable or provide for the connection and use of the display device(s) 624a-624n by the computing device 600. For example, the computing device 600 may include any type and/or form of video adapter, video card, driver, and/or library to interface, communicate, connect or otherwise use the display device(s) 624a-624n. In one embodiment, a video adapter may include multiple connectors to interface to the display device(s) 624a-624n. In other embodiments, the computing device 600 may include multiple video adapters, with each video adapter connected to the display device(s) 624a-624n. In some embodiments, any portion of the operating system of the computing device 600 may be configured for using multiple displays 624a-624n. One ordinarily skilled in the art will recognize and appreciate the various ways and embodiments that a computing device 600 may be configured to have one or more display devices 624a-624n.

In further embodiments, an I/O device 630 may be a bridge between the system bus 650 and an external communication bus, such as a USB bus, an Apple Desktop Bus, an RS-232 serial connection, a SCSI bus, a Fire Wire bus, a FireWire 800 bus, an Ethernet bus, an AppleTalk bus, a Gigabit Ethernet bus, an Asynchronous Transfer Mode bus, a FibreChannel bus, a Serial Attached small computer system interface bus, a USB connection, or a HDMI bus.

A computing device 600 of the sort depicted in FIGS. 6A and 6B may operate under the control of an operating system, which control scheduling of tasks and access to system resources. The computing device 600 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: Android, produced by Google Inc.; WINDOWS 7 and 8, produced by Microsoft Corporation of Redmond, Washington; MAC OS, produced by Apple Computer of Cupertino, California; WebOS, produced by Research In Motion (RIM); OS/2, produced by International Business Machines of Armonk, New York; and Linux, a freely-available operating system distributed by Caldera Corp. of Salt Lake City, Utah, or any type and/or form of a Unix operating system, among others.

The computer system 600 can be any workstation, telephone, desktop computer, laptop or notebook computer, server, handheld computer, mobile telephone or other portable telecommunications device, media playing device, a gaming system, mobile computing device, or any other type and/or form of computing, telecommunications or media device that is capable of communication. The computer system 600 has sufficient processor power and memory capacity to perform the operations described herein.

In some embodiments, the computing device 600 may have different processors, operating systems, and input devices consistent with the device. For example, in one embodiment, the computing device 600 is a smart phone, mobile device, tablet or personal digital assistant. In still other embodiments, the computing device 600 is an Android-based mobile device, an iPhone smart phone manufactured by Apple Computer of Cupertino, California, or a Blackberry or WebOS-based handheld device or smart phone, such as the devices manufactured by Research In Motion Limited. Moreover, the computing device 600 can be any workstation, desktop computer, laptop or notebook computer, server, handheld computer, mobile telephone, any other computer, or other form of computing or telecommunications device that is capable of communication and that has sufficient processor power and memory capacity to perform the operations described herein.

Although the disclosure may reference one or more “users”, such “users” may refer to user-associated devices or stations (STAs), for example, consistent with the terms “user” and “multi-user” typically used in the context of a multi-user multiple-input and multiple-output (MU-MIMO) environment.

Although examples of communications systems described above may include devices and APs operating according to an 802.11 standard, it should be understood that embodiments of the systems and methods described can operate according to other standards and use wireless communications devices other than devices configured as devices and APs. For example, multiple-unit communication interfaces associated with cellular networks, satellite communications, vehicle communication networks, and other non-802.11 wireless networks can utilize the systems and methods described herein to achieve improved overall capacity and/or link quality without departing from the scope of the systems and methods described herein.

It should be noted that certain passages of this disclosure may reference terms such as “first” and “second” in connection with devices, mode of operation, transmit chains, antennas, etc., for purposes of identifying or differentiating one from another or from others. These terms are not intended to merely relate entities (e.g., a first device and a second device) temporally or according to a sequence, although in some cases, these entities may include such a relationship. Nor do these terms limit the number of possible entities (e.g., devices) that may operate within a system or environment.

It should be understood that the systems described above may provide multiple ones of any or each of those components and these components may be provided on either a standalone machine or, in some embodiments, on multiple machines in a distributed system. In addition, the systems and methods described above may be provided as one or more computer-readable programs or executable instructions embodied on or in one or more articles of manufacture. The article of manufacture may be a floppy disk, a hard disk, a CD-ROM, a flash memory card, a PROM, a RAM, a ROM, or a magnetic tape. In general, the computer-readable programs may be implemented in any programming language, such as LISP, PERL, C, C++, C#, PROLOG, or in any byte code language such as JAVA. The software programs or executable instructions may be stored on or in one or more articles of manufacture as object code.

While the foregoing written description of the methods and systems enables one of ordinary skill to make and use what is considered presently to be the best mode thereof, those of ordinary skill will understand and appreciate the existence of variations, combinations, and equivalents of the specific embodiment, method, and examples herein. The present methods and systems should therefore not be limited by the above described embodiments, methods, and examples, but by all embodiments and methods within the scope and spirit of the disclosure.

We claim:

1. A method for determining drug inhibitory concentrations, comprising:
receiving, by one or more computing devices, a plurality of images of cells treated with a candidate drug, the plurality of images comprising subsets of one or more images, each subset corresponding to a different concentration of the candidate drug;
classifying, by the one or more computing devices, each image of the plurality of images as treated or untreated;
calculating, by the one or more computing devices, a classification accuracy for each subset of the plurality of images;
determining, by the one or more computing devices, a concentration of the candidate drug corresponding to a subset of the plurality of images having a greatest change in classification accuracy relative to a subset corresponding to a next-highest or next-lowest concentration; and
identifying, by the one or more computing devices, the determined concentration as corresponding to an inhibitory concentration.

2. The method of claim 1, further comprising receiving, by the one or more computing devices, a second plurality of images of untreated cells.

3. The method of claim 2, further comprising dividing the plurality of images of treated cells and the second plurality of images of untreated cells into a first set of training data and a second set of test data.

4. The method of claim 1, further comprising augmenting the plurality of images of cells treated with the candidate drug by creating additional images via one or more image manipulations of the received images.

5. The method of claim 1, further comprising filtering, by the one or more computing devices, each image of the plurality of images to identify edges within each image.

6. The method of claim 5, wherein filtering each image of the plurality of images further comprises applying a Sobel filter to each image.

7. The method of claim 1, wherein classifying each image of the plurality of images as treated or untreated comprises providing each image to one or more vision transformers executed by the one or more computing devices.

8. The method of claim 1, wherein calculating the classification accuracy for each concentration of the candidate drug further comprises comparing the classification of each image to a predetermined treatment classification for the image.

9. The method of claim 1, wherein determining the concentration of the candidate drug corresponding to the subset of the plurality of images having the greatest change in classification accuracy relative to a subset corresponding to a next-highest or next-lowest concentration comprises determining a Hill curve corresponding to the calculated classification accuracies; and identifying a concentration of the candidate drug corresponding to a portion of the Hill curve having a greatest slope.

10. The method of claim 1, wherein determining the concentration of the candidate drug corresponding to the subset of the second plurality of images having the greatest change in classification accuracy relative to a subset corresponding to a next-highest or next-lowest concentration comprises determining a slope between each pair of adjacent concentrations and corresponding classification accuracies and identifying a concentration of the candidate drug corresponding to a maximum determined slope.

11. A system for determining drug inhibitory concentrations, comprising:
one or more computing devices comprising one or more processors configured to:
receive a plurality of images of cells treated with a candidate drug, the plurality of images comprising subsets of one or more images, each subset corresponding to a different concentration of the candidate drug,
classify each image of the plurality of images as treated or untreated,
calculate a classification accuracy for each subset of the plurality of images,
determine a concentration of the candidate drug corresponding to a subset of the plurality of images having a greatest change in classification accuracy relative to a subset corresponding to a next-highest or next-lowest concentration, and
identify the determined concentration as corresponding to an inhibitory concentration.

12. The system of claim 11, wherein the one or more processors are further configured to receive a second plurality of images of untreated cells.

13. The system of claim 12, wherein the one or more processors are further configured to divide the plurality of images of treated cells and the second plurality of images of untreated cells into a first set of training data and a second set of test data.

14. The system of claim 11, wherein the one or more processors are further configured to augment the plurality of images of cells treated with the candidate drug by creating additional images via one or more image manipulations of the received images.

15. The system of claim 11, wherein the one or more processors are further configured to filter each image of the plurality of images to identify edges within each image.

16. The system of claim 15, wherein filtering each image of the plurality of images further comprises applying a Sobel filter to each image.

17. The system of claim 11, wherein the one or more processors are further configured to apply one or more vision transformers to each image.

18. The system of claim 11, wherein the one or more processors are further configured to compare the classification of each image to a predetermined treatment classification for the image.

19. The system of claim 11, wherein the one or more processors are further configured to determine a Hill curve corresponding to the calculated classification accuracies; and identify a concentration of the candidate drug corresponding to a portion of the Hill curve having a greatest slope.

20. The system of claim 11, wherein the one or more processors are further configured to determine a slope between each pair of adjacent concentrations and corresponding classification accuracies and identify a concentration of the candidate drug corresponding to a maximum determined slope.
